(19) United States
(12) Patent Application Publication
(10) Pub. No.: US 2015/0374972 A1
(43) Pub. Date: Dec. 31, 2015

(54) PATCH DEVICE

(71) Applicant: YBRAIN INC., Seoul (KR)

(72) Inventor: Kiwon LEE, Daejeon (KR)

(21) Appl. No.: 14/755,787

(22) Filed: Jun. 30, 2015

(30) Foreign Application Priority Data
Jun. 30, 2014 (KR) 10-2014-0080714

(51) Int. Cl. A61N 1/04; A61B 5/00; A61N 1/36; A61B 5/0478; A61N 1/30

(52) U.S. Cl. CPC A61N 1/0492; A61B 5/0478; A61N 1/303; A61N 1/36025; A61B 5/6833; A61B 2562/0209

(57) ABSTRACT

The present disclosure relates to a patch device attached to a head to apply electrical stimulation to a brain, comprising, in accordance with one exemplary embodiment of the present invention, a flexible board comprising a first side and a second side placed on the opposite of the first side, bendable along with a curve of the head; a stimulation control circuit formed on the first side of the flexible board and controlling a current applied to a plurality of adhesion portion; a cover layer formed on the stimulation control circuit in order to cover the first side; and a plurality of adhesion portion adhered to the head when the patch device is attached to the head, formed on the second side of the flexible board separately from each other for insulation.

### CROSS-REFERENCE TO RELATED APPLICATION

This application claims the priority of the Korean Patent Application No. 10-2014-0080714, filed on Jun. 30, 2014 in the Korean Intellectual Property Office (KIPO), and all the benefits accruing therefrom under 35 U.S.C. §119, the contents of which in their entirety are herein incorporated by reference.

### TECHNICAL FIELD

The present invention is related to a patch device, and particularly, to a patch device adhered to a head to apply electrical stimulation to a brain or to measure the brainwaves therefrom.

### BACKGROUND ART

It is known that technology for electrical stimulation of the brain using Transcranial Direct Current Stimulation (TDCS) is effective for improvement of recognition ability and for treatment of mental disease such as depression and ADHD (Attention Deficit Hyperactivity Disorder).

Therefore, if the technology is available in everyday life, the continuous treatment of mental disease would be possible through either activation or refrainment of the nerves, and the brain function could be improved.

### RELATED ART DOCUMENT

### Patent Document

(Patent 1) Patent Laid-Open Publication No. 10-2011-0064071

### DISCLOSURE OF INVENTION

### Technical Problem

The conventional electrical stimulation apparatus is mainly designed for the patch to be adhered to a ductile strap or a head cap, and then for the strength of the electrical stimulation to be manually controlled. The brainwave measuring apparatus is also designed for the patch to be adhered to a ductile strap or a head cap to measure brainwaves. Therefore, the conventional electrical stimulation apparatus and the brainwave measuring apparatus have only been intermittently used by experts; the ordinary people unfamiliar with the expertise in the structure and the position of the brain, and the acceptable amount of electric current cannot use the electrical stimulation apparatus or the brainwave measuring apparatus in everyday life due to a danger to safety accidents caused by mal-operation.

Moreover, the conventional electrical stimulation apparatus and the brainwave measuring apparatus are inconvenient to use due to a great size, and lack of aesthetics and activity due to a high price.

The technical issue the present invention seeks in order to resolve the above mentioned problems is to provide a patch device which ordinary people without expertise can also use safely in everyday life for applying electrical stimulation and for measuring brainwaves.

Another technical issue the present invention seeks in order to resolve the above problem is to provide a patch device inexpensive and excellent in portability, wearability, and aesthetics by a simple structure.

Further technical issue the present invention seeks in order to resolve the above problem is to provide a patch device which performs application of electrical stimulation with medication, monitors the effect of medication, and controls the amount of medication to be used.

The technical issue of the present invention is not limited to those mentioned above; the other technical issues not mentioned herein will be clearly understandable for the ordinary skilled in the art by the description set forth herein.

### Advantageous Effects

The patch device, according to the present invention, is easy to use, thus the ordinary user without expertise can also use it safely in everyday life.

The patch device, according to the present invention, is excellent in portability wearability, and aesthetics by a simple structure, and is inexpensive

Moreover, the patch device, according to the present invention, can perform the application of electrical stimulation with medication, monitor the effect of medication, and control the amount of medication to be used.

### BRIEF DESCRIPTION OF THE DRAWINGS

FIG. 1 is a diagram illustrating an outline of the structure of the patch device (1) according to one exemplary embodiment of the present invention.

FIG. 2 is a perspective diagram illustrating a condition of the patch device (1) of FIG. 1 adhered to the head (H).

FIG. 3 is a diagram illustrating a contact of an adhesion portion (40) to an electrode portion (60) included in the patch device (1) of FIG. 1.

FIG. 4 is a diagram illustrating an outline of the structure of the patch device (2) according to another exemplary embodiment of the present invention.

FIG. 5 is a diagram illustrating an outline of the structure of the patch device (3).

FIG. 6 is a diagram illustrating an outline of structure of an integrated circuit (70).

FIG. 7 is a diagram illustrating an outline of the structure of the patch device (4) according to the forth exemplary embodiment of the present invention.

FIG. 8 is a diagram illustrating an outline of the structure of the patch device (5) according to the fifth exemplary embodiment of the present invention.

FIG. 9 is a diagram illustrating an outline of the structure of the flexible board (10) included in the patch device according to the exemplary embodiments.

FIG. 10 is a diagram illustrating a combination of the flexible board and the circuit, included in the patch device according to the exemplary embodiments of the present invention.

### BEST MODE OF CARRYING OUT THE INVENTION

A desirable exemplary embodiment of the present invention will be described in detail hereinafter in reference with the accompanied drawings. The advantage and characteristic, and a method to achieve them will be obvious upon detailed description of the exemplary embodiments as well as accompanied drawings which follow. However, the present invention is not limited to the exemplary embodiments set forth herein, but can be embodied in many different forms. The exemplary embodiments are provided only to render the present disclosure complete and the scope of the present invention comprehensible to those having ordinary skill in the art. The present invention is only to be defined by the claims. A same reference mark used refers to a same element throughout the description.

Without separate definitions, all terms (including technical and scientific terms) used in the present description may be used for the meanings commonly understandable to those having ordinary skill in the art. In addition, the terms generally used and having definitions in dictionary, unless otherwise defined obviously in particular, should not be ideally or exaggeratedly interpreted.

The terms used in the present description are to explain the exemplary embodiments, not to limit the present invention thereto. In the present description, a singular form of word also includes a plural form thereof unless otherwise noted. The term “comprises” and/or “comprising” is not excluding the meaning that one or more elements other than the said element may exist or be added.

The explanation on the patch device according to the exemplary embodiment of the present invention will be followed in reference with the drawings.

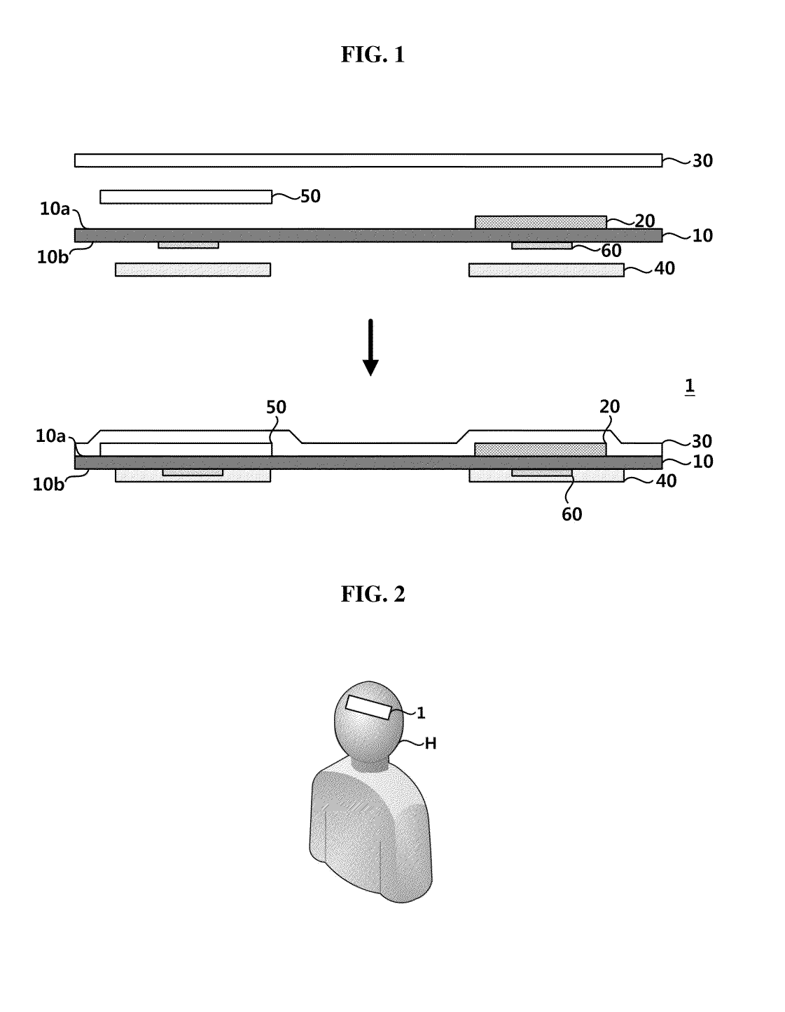

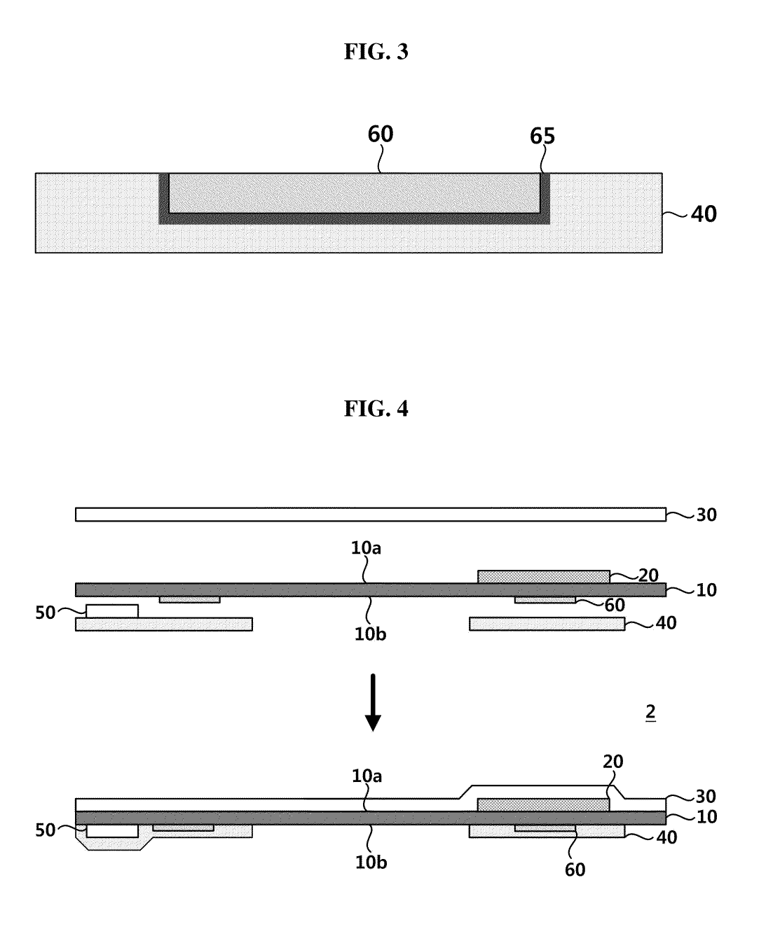

First of all, in reference with FIG. 1 to FIG. 3, a patch device (1) according to one exemplary embodiment of the present invention will be described. FIG. 1 is a diagram illustrating an outline of the structure of the patch device (1) according to one exemplary embodiment of the present invention. FIG. 2 is a perspective diagram illustrating a condition of the patch device (1) of FIG. 1 adhered to the head (H). FIG. 3 is a diagram illustrating a contact of an adhesion portion (40) to an electrode portion (60) included in the patch device (1) of FIG. 1.

In reference with FIG. 1 and FIG. 2, the patch device (1) according to one exemplary embodiment of the present invention, worn on the head (H) of a user, can apply electrical stimulation to the brain of the user. Particularly, just worn on the head (H) of the user, the patch device (1) can apply electrical stimulation to the targeted area of the brain by electrical current flowing to the head (H) through the adhesion portion (40) of the patch.

In reference with FIG. 1, the patch device (1) may include a flexible board (10), a stimulation control circuit (20), a cover layer (30), and a plurality of adhesion portion (40). In some exemplary embodiments, a power supply portion (50) and a plurality of electrode portions (60) can be further included. As each element illustrated in FIG. 1 is not an essential element, the patch device (10) may be embodied with either more elements or less.

A flexible board (10), a ductile circuit board, may include a first side (10a) and a second side (10b), and the second side (10b) may be positioned in the opposite of the first side (10a). The flexible board (10) can provide a side on which a circuit and a part can be formed. Particularly, a circuit can be formed both on the first side (10a) and the second side (10b) while the place is not limited thereto. Since the flexible board (10) can bend with the curve of the head (H), the patch device (1) according to one exemplary embodiment of the present invention can be adhered to the head (H) for the use.

In some exemplary embodiments, an adhesive formed on the second side (10b) of the flexible board (10) can help the patch device (1) adhering to the head (H) of the user, but the medium is not limited thereto.

A stimulation control circuit (20) can be formed on the first side (10a) of the flexible board (10) and control the electrical current to be applied to a plurality of the adhesion portions (40). That is, when the patch device (10) is attached to the head (H) to apply electrical stimulation to the brain, the stimulation control circuit (20) can control the electrical current to be applied to a plurality of the adhesion portions (40) to the level of amount pre-set or to the amount less than the level, preventing from a safety accident which may occur due to overcurrent.

In order for this, the stimulation control circuit (20) can be formed only with current limiting diode, particularly with the passive element current limiting diode, but the element is not limited thereto.

A cover layer (30) can cover the first side (10a) of the flexible board (10). For example, the cover layer (30) can cover the element formed on the first side (10a) of the flexible board (10), including the stimulation control circuit (20). Accordingly, the integrated structure, wherein the circuit is not exposed on the flexible board (10), can be formed, enabling the manufacturing process to be simplified and the miniaturization with low cost.

Meanwhile, the cover layer (30) can be formed with an insulating material; therefore, the circuit element on the flexible board (10) can be protected by insulation, preventing from a safety accident such as electric shock or leakage. However, the material is not limited as long as it is insulating.

In some exemplary embodiments, the cover layer (30) painted with skin color, the patch device (1) attached to the head (H) does not draw attention. As necessary, the cover layer (30) can be painted with different colors, or an image can be included on the exterior of the cover layer (30), improving the aesthetics of the patch device (1).

A plurality of the adhesion portions (40) can be adhered to the head (H) of the user when the patch device (1) is attached thereto. Particularly, a plurality of the adhesion portions (40) can be formed on the second side (10b) of the flexible board (10), and a plurality of the adhesion portions (40) can be separated from each other in order to be insulated. A plurality of the adhesion portions (40) may be formed at regular intervals on the second side (10b) of the flexible board (10).

On the other hand, since a plurality of the adhesion portions (40), formed on the second side (10b) of the flexible board (10), are replaceable, a plurality of used adhesion portions (40) can be removed, and a plurality of new adhesion portions (40) can be attached on the second side (10b) of the flexible board (10), enabling the patch device (1) to be used again.

At least a part of a plurality of the adhesion portion (40) may include a conductive hydrogel. Since the adhesion portion (40) includes the hydrogel, the electrical stimulation signal (or the brainwave signal) can be easily transmitted between the patch device (1) and the head (H) as the impedance between the patch device (1) and the scalp decreases.

In some exemplary embodiments, at least a part of a plurality of the adhesion portion (40) may include a general medication such as an excelon or medication available for iontophoresis. Therefore, the patch device (1) according to the exemplary embodiment can apply electrical stimulation with injection of medication; thus, various synergy effects can be achieved, such as the improvement of neuro-transmission between the brain nerves by medication, and the increase of frequency of neuro-transmission activity of the brain nerves by electrical stimulation.

In addition, at least a part of a plurality of the adhesion portions (40) according to other exemplary embodiments may include a conductive hydrogel including medication.

A power supply portion (50) can supply the power generating electrical current necessary for electrical stimulation. The power supply portion (50), as a flexible power supply portion (50), can be formed on the first side (10a) of the flexible board (10) while the position is not limited thereto; the power supply portion (50) can also be formed on the second side (10b) of the flexible board (10).

Since the power supply portion (50) has ductility, the patch device (1) according to one exemplary embodiment can bend with the curve of the head (H), making it convenient to use with attached to the head (H).

Meanwhile, the power supply portion (50) can be a rechargeable power supply portion (50), while the form is not limited thereto, and also can be a disposable power supply portion (50) replaceable as necessary.

Besides, in case the power supply portion (50) is a flexible battery, the power supply portion (50) can be formed with at least one flexible battery connected in series. The neighboring flexible battery can be connected by solder, isotropic/anisotropic conductive adhesive, or isotropic/anisotropic conductive film.

A plurality of electrode portions (60) can be positioned separately on the second side (10b) of the flexible board (10). Each of a plurality of adhesion portions (40) can be formed on a plurality of electrode portions (60). Accordingly, a plurality of adhesion portions (40) can receive necessary current applied from a plurality of electrode portions (60). The electrode portion (60) has the positive and negative poles, separately, to run direct current, but the structure is not limited thereto.

In reference with FIG. 3, a plurality of electrode portions (60) may have the surface treated. For example, the surface of a plurality of electrode portions (60) may be covered with a coating layer (65) which is made of conductive and noninvasive solid. A plurality of adhesion portions (40) may be formed on a plurality of electrode portions (60) which has the surface treated. Therefore, even though the adhesion portion (40) includes hydro-gel with electrolyte, the corrosion of the electrode portion (60) can be prevented due to the coating layer (65) treated on the surface of the electrode portion (60).

Moreover, because the surface of electrode portion (60) is treated with the coating layer (65), the corrosion of the electrode portion (60) by foreign substance such as perspiration come out from the scalp of the user can be prevented when the user moving with the patch device (1) attached to his/her head (H).

Therefore, according to one exemplary embodiment of the present invention, the patch device (10) can be safely used in everyday life by an ordinary user without expertise as it includes the stimulation control circuit (20) and has a simple way of use. And the patch device (1) of the present exemplary embodiment can be excellent in portability, wearability, and aesthetics, and inexpensive by a simple structure; and can apply electrical stimulation with medication.

The patch device (1) according to one exemplary embodiment of the present invention is convenient to use because the electrical stimulation begins as it is automatically triggered by the contact of a plurality of adhesion portion (40) to scalp.

In reference with FIG. 4, a patch device (2) according to another exemplary embodiment of the present invention will be explained thereinafter. FIG. 4 is a diagram illustrating an outline of the structure of the patch device (2) according to another exemplary embodiment of the present invention. The difference from the patch device (1) according to one exemplary embodiment will be mainly described.

The patch device (1) according to one exemplary embodiment of FIG. 1 may have a power supply portion (50) and an adhesion portion (40) as a separate composition. On the other hand, in reference with FIG. 4, at least a part of a plurality of adhesion portions (40) of the patch device (2) may include a flexible power supply portion (50). That is, the adhesion portion (40) and the flexible power supply portion (50) can be combined as an integral form.

Therefore, the consumable elements such as the adhesion portion (40) and the flexible power supply portion (50) of the patch device (2) can be replaced at once, making it convenient for the use of the patch device (2). Furthermore, the integration of the adhesion portion (40) and the flexible power supply portion (50) can promote a miniaturization of the patch device (2)

It is obvious to the ordinary skilled in the art that technical features of the patch device (2) according to the second exemplary embodiment of the present invention, explained herein in reference with FIG. 4, can also be applied to the patch device according to the following third to fifth exemplary embodiments of the present invention.

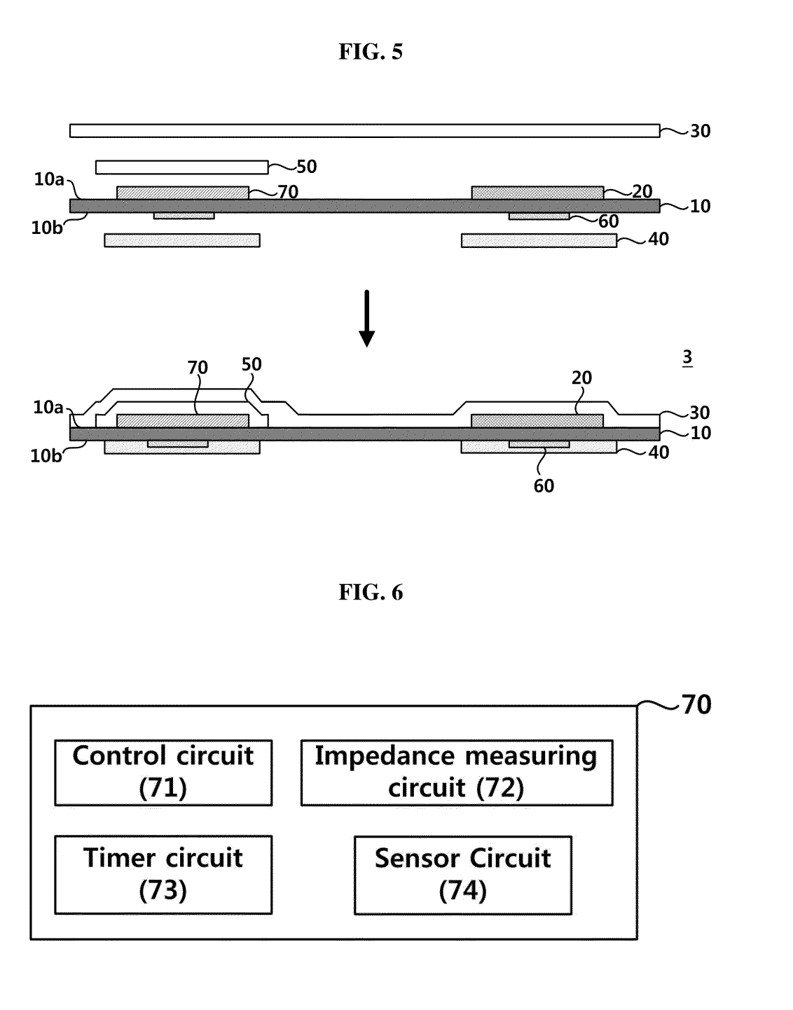

Below, the patch device (3) according to the third exemplary embodiment of the present invention will be explained in reference with FIGS. 5 and 6. FIG. 5 is a diagram illustrating an outline of the structure of the patch device (3), and FIG. 6 is a diagram illustrating an outline of structure of an integrated circuit (70). The difference from the patch device (1) according to one exemplary embodiment will be mainly explained.

In reference with FIG. 5, the patch device (3) may include an integrated circuit (70) formed on the first side (10a) of the flexible board (10). For example, the active side of the integrated circuit (70) can be connected to the first side (10a) of the flexible board (10). The integrated circuit (70) may be made of silicone, but the material is not limited thereto.

In reference with FIG. 6, the integrated circuit (70) may include a control circuit (71), an impedance measuring circuit (72), a timer circuit (73), and a sensor circuit (74). However, each element described in FIG. 6 is not an essential element, the integrated circuit (70) can also be embodied with more elements or less.

A control circuit (71) can control the operation of the patch device (3). The control circuit (71) can control, as necessary, the electrode portion (60), the stimulation control circuit (20), an amplification circuit (90) and a medication pouch (80) which will be explained thereinafter, as well as the element of the integrated circuit (70).

An impedance measuring circuit (72) can measure the impedance by the adhesion portion (40). Particularly, when the user has the patch device (3) attached, the impedance measuring circuit (72) can measure the bio-impedance or the electrode impedance through the adhesion portion (40) as it is contacted to the head (H). Due to the value of impedance predictable in case the user has the patch device (3) properly attached, and so the appropriate contact has been made, it can be judged whether or not the patch device (3) is properly attached and adhered to the targeted area of the adhesion portion (40) on the basis of the result of measurement drawn by the impedance measuring circuit (72).

Therefore, the control circuit (71) can control applying of the electrical current flowing into the adhesion portion (40) on the basis of the result of measurement drawn by the impedance measuring circuit (72). Accordingly, the electrical stimulation by the patch device (3) can begin.

According to the third exemplary embodiment of the present invention, the patch device (3) can be safely used by the user because the electrical stimulation can begin only in case the patch device (3) is properly attached to the head (H) of the user.

A timer circuit (73) can count the necessary time in order for the control of the patch device (3). And, a sensor circuit (74) can detect the trembling or the pressure changes of the patch device (3). The sensor circuit (74) may include a gyro sensor or a decompression sensor, but the element is not limited thereto.

A control circuit (71) can control the timer circuit (73) to count the pre-set time, after the electrical stimulation begins on the basis of the result of measurement drawn by the impedance measuring circuit (72) or the result of detection drawn by the sensor circuit (74). And, the control circuit (71) can control no more current to be applied to flow into the adhesion portion (4) of the patch device (3) when the timer circuit (73) detects the pre-set time limit is exceeded. According to the third exemplary embodiment of the present invention, the patch device (3) can be safely used because the electrical stimulation automatically stops after the pre-set time limit.

In addition, when the trembling or the pressure changes on the patch device (3) are detected by the sensor circuit (74), the control circuit (71) can control no more current to be applied to flow into the adhesion portion (40) of the patch device (3). The trembling or the pressure change on the patch device (3) may be caused by the user's trying to remove the patch device (3); therefore, by stopping the electrical stimulation of the patch device (3), the user safety may be promoted.

It is obvious to the ordinary skilled in the art the technical features of the patch device (3), previously described in reference with FIG. 6, according to the third exemplary embodiment of the present invention can be applied to the patch device (5) according to the fifth exemplary embodiment of the present invention which follows within a range that the technical idea is not conflicting with each other.

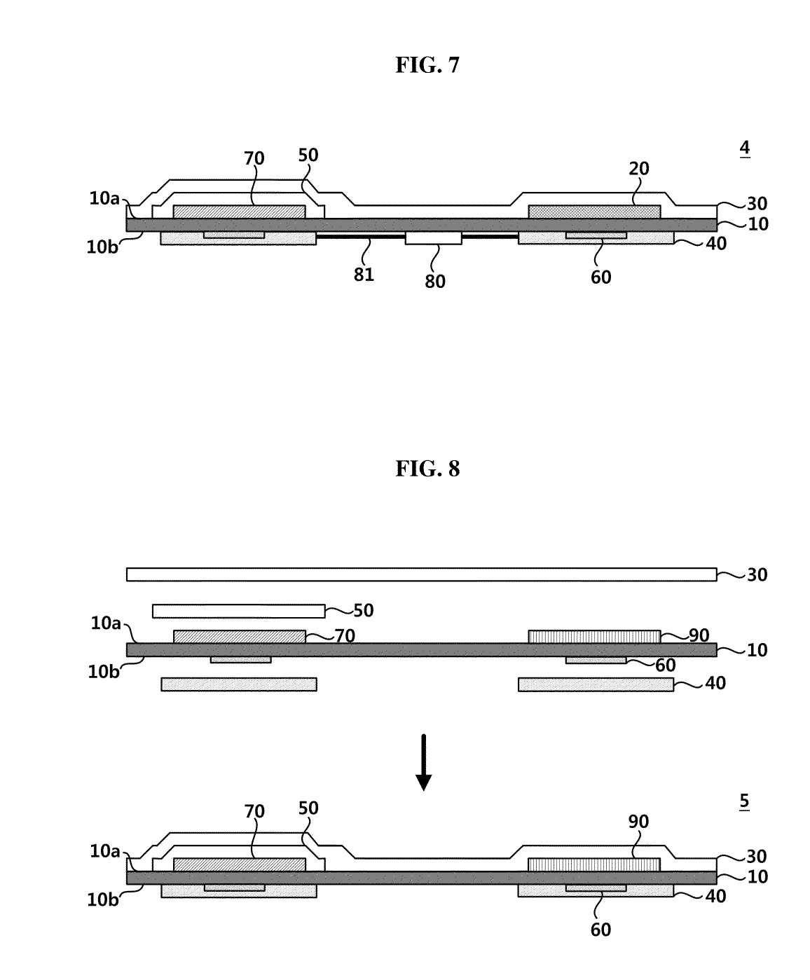

In reference with FIG. 7, a patch device (4) according to the forth exemplary embodiment of the present invention will be explained hereinafter. FIG. 7 is a diagram illustrating an outline of the structure of the patch device (4) according to the forth exemplary embodiment of the present invention. The difference from the patch device (3) according to the third exemplary embodiment will be mainly described.

In reference with FIG. 7, the patch device (4) may include a medication pouch (80) providing the medication connected to at least a part of a plurality of the adhesion portion (40). The medication of the medication pouch (80) can be provided through a connecting pipe (81) which is connected to the medication pouch (80) and the adhesion portion (40). The medication stored in the medication pouch (80) may be a general medication such as an excelon or the medication available for iontophoresis, but the kind is not limited thereto.

The amount of medication supplied from the medication pouch (80) can be controlled depending on the amount of current transferred by a plurality of adhesion portions (40) or the brainwave received therefrom. The amount of medication supply of the medication pouch (80) can be controlled by the control circuit (71), but the controller is not limited thereto.

The patch device (4) according to the forth exemplary embodiment of the present invention can promote the user safety because the amount of medication supply of the medication pouch (80) can be controlled.

In addition, since the patch device (4) according to the forth exemplary embodiment of the present invention can apply electrical stimulation with injection of medication, various synergy effects can be achieved, such as the improvement of neurotransmission between the brain nerves by medication, and the increase of frequency of neurotransmission activity of the brain nerves by the electrical stimulation.

In addition, the patch device (4) according to the forth exemplary embodiment of the present invention can detect a side effect of medication, such as dermal edema or infection, by detecting the change of impedance.

The effect of medication can be monitored as the brainwave generated by injection of medication can be measured according to the patch device (4) of the forth exemplary embodiment of the present invention.

It is obvious to the ordinary skilled in the art the technical features of the patch device (4) according to the forth exemplary embodiment of the present invention described in reference with FIG. 7 can be applied to the patch device according to the first and second exemplary embodiments, and the fifth exemplary embodiment which follows. According to the brainwaves received from a plurality of the adhesion portions (40), the amount of medication supplied by the medication pouch (80) can be controlled.

The patch device (5) according to the fifth exemplary embodiment of the present invention will be explained hereinafter in reference with FIG. 8. FIG. 8 is a diagram illustrating an outline of the structure of the patch device (5) according to the fifth exemplary embodiment of the present invention. Only the difference from the patch device (3) according to the third exemplary embodiment will be described.

While the patch device (3) of FIG. 5 is an electrical stimulation apparatus including the stimulation control circuit (20), the patch device (5) of FIG. 8 may be a brainwave measuring apparatus including an amplifying circuit (90). In reference with FIG. 8, the patch device (5) which measures the brainwave from the brain by being attached to the head (H) may include a flexible board (10) including a first side (10a) and a second side (10b) placed on the opposite of the first side (10a), an amplifying circuit (90) formed on the first side (10a) of the flexible board (10) and amplifies the brainwave received from a plurality of the adhesion portions (40), a cover layer (30) formed on the amplifying circuit to cover the first side (10a), and a plurality of adhesion portions (40) adhered to the head (H) when the patch device (5) is attached to the head (H), and formed on the second side (10b) of the flexible board (10), separately from each other for insulation.

The patch device (5) according to the fifth exemplary embodiment of the present invention can monitor the brainwave of the user as the adhesion portion (40) receives the brainwave, and the amplifying circuit (90) amplifies the received brainwaves. The brainwaves can be measured as the electrode portion (60) is divided into a reference electrode and a measuring electrode, but the method is not limited thereto.

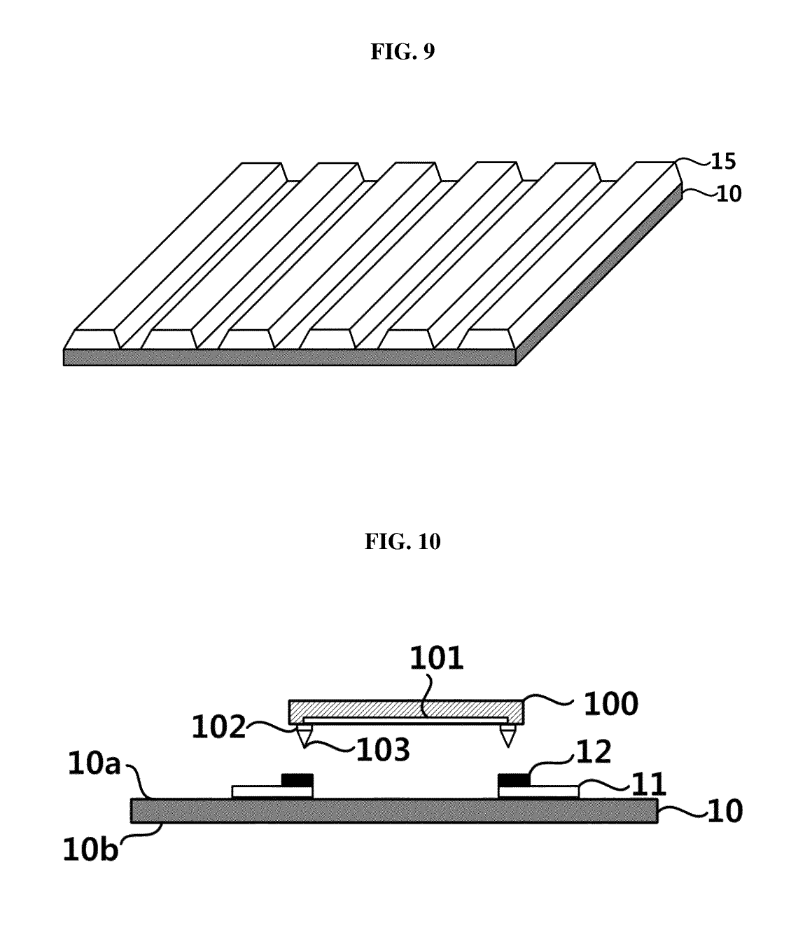

In reference with FIG. 9, the flexible board (10) included in the patch device according to the exemplary embodiments of the present invention will be explained hereinafter. FIG. 9 is a diagram illustrating an outline of the structure of the flexible board (10) included in the patch device according to the exemplary embodiments. Only the difference from the flexible board (10) of FIG. 1 will be mainly described.

In reference with FIG. 9, a stiffener (15) can be formed on the flexible board (10) of the patch device. The stiffener (15) can prevent a specific circuit and a part, vulnerable to bending, from damage when the flexible board (10) is bent. In case the stiffener (15) is formed with a certain pattern, the damage on the circuit and the part vulnerable to bending is prevented while the parts safe to be bent continues to bend, and at the same time, the aesthetics of the exterior can be promoted. The circuit or part can be formed on the stiffener (15), and as necessary, can be formed between the neighboring stiffeners or beneath the stiffener (15).

Below, the combination of the flexible board and the circuit, included in the patch device according to the exemplary embodiments of the present invention, will be explained. FIG. 10 is a diagram illustrating a combination of the flexible board and the circuit, included in the patch device according to the exemplary embodiments of the present invention.

In reference with FIG. 10, a circuit (11) can be formed on the first side (10a) of the flexible board (10), and a pad (12) can be formed on the circuit (11) to be combined with another part or circuit. As necessary, the circuit (11) and the pad (12) can be formed on the second side (10b) of the flexible board (10).

A part (100) can be combined on the flexible board (10). Particularly, the part (100) can be combined on the flexible board (10) in a state that an active area (101) of the part (100) is facing the flexible board (10). The part (100) may be a kind of circuit, but the kind is not limited as long as it can be combined on the flexible board (10).

Particularly, an I/O pad (102) can be formed on the side wherein the active area (101) of the part (100) is formed in order to be electrically connected with the active area (101), and a bump (103) can be formed on the I/O pad (102). That is, the bump (103) and the active area (101) can be electrically connected by the I/O pad (102). As the bump (103) is connected with the pad (12) of the flexible board (10), the part (100) and the flexible board (10) can be electrically connected. Meanwhile, the bump (103) may be an Au stud, and the pad (12) may be formed with Sn. In case the bump (103) is a micro solder, the material of the pad (12) may not be limited. In addition, in case the bump (103) is formed with Au, the bump (103) and the pad (12) can be connected by the non-conductive adhesive/film. Also, the bump (103) and the pad (12) can be connected by the anisotropic conductive adhesive/film. But such combination is only exemplary, not limiting the exemplary embodiment of the present invention.

It is clearly understandable for those having ordinary skill in the art the present invention can be embodied in various forms, other than the exemplary embodiments set forth herein, without changes in its technical idea or essential characteristic. The exemplary embodiments described herein are only for the purpose of exemplifying the present invention in all aspects, not of limiting the scope of the present invention.

[Description for Reference Numerals]

| | |
|---|---|
| 1: Patch device | H: Head |
| 10: Flexible board | 10a, 10b: First side, Second side |
| 11: Circuit | 12: Pad |

| 15: Stiffener | |
|---|---|
| 20: Stimulation control circuit | 30: Cover layer |
| 40: Adhesion portion | 50: Power Supply portion |
| 60: Electrode portion | 65: Coating layer |
| 70: Integrated circuit | 71: Control circuit |
| 72: Impedance measuring circuit | 73: Timer circuit |
| 74: Censor circuit | 80: Medication pouch |
| 81: Connecting pipe | 90: Amplifying circuit |
| 100: Part | 101: Active area |
| 102: I/O pad | 103: Bump |

1. A patch device attached to a head to apply electrical stimulation to a brain,
comprising a flexible board comprising a first side and a second side placed on the opposite of the first side, bendable along with a curve of the head;
a stimulation control circuit formed on the first side of the flexible board and controlling a current applied to a plurality of adhesion portion;
a cover layer formed on the stimulation control circuit in order to cover the first side; and
a plurality of adhesion portion adhered to the head when the patch device is attached to the head, formed on the second side of the flexible board separately from each other for insulation,
wherein at least a part of the flexible board is exposed by a plurality of the adhesion portions, and adhered to the head when the patch device is attached to the head.

2. A patch device attached to a head to measure brainwave, comprising
a flexible board comprising a first side and a second side placed on the opposite of the first side, bendable along with a curve of the head;
an amplifying circuit formed on the first side of the flexible board and measuring brainwave received from a plurality of adhesion portion;
a cover layer formed on the amplifying circuit to cover the first side; and
a plurality of adhesion portion adhered to the head when the patch device is attached to the head, and formed on the second side of the flexible board separately from each other for insulation,
wherein at least a part of the flexible board is exposed by a plurality of the adhesion portions, and adhered to the head when the patch device is attached to the head.

3. According to claim 1,
the patch device, formed on the first side of the flexible board, wherein an active side further comprises an integrated circuit connected to the first side.

4. According to claim 3,
the patch device,
wherein the integrated circuit comprises a control circuit controlling an operation of the patch device, and further comprises a timer circuit or a sensor circuit; and
the control circuit controls no more current to be applied to the adhesion portion when the timer circuit detects the pre-set time limit is exceeded, or the trembling or pressure change is detected by the sensor circuit.

5. According to claim 3,
the patch device,
wherein the integrated circuit comprises a control circuit controlling an operation of the patch device, an impedance measuring circuit measuring impedance through the adhesion portion, and
the control circuit controls applying of the current flowing into the adhesion portion on the basis of the result of measurement by the impedance measuring circuit.

6. According to claim 2,
the patch device, formed on the first side of the flexible board, further comprising an integrated circuit wherein an active side is connected to the first side.

7. According to claim 1,
the patch device, wherein at least a part of a plurality of the adhesion portions comprises a conductive hydrogel.

8. According to claim 7,
the patch device, wherein at least a part of a plurality of the adhesion portions comprises a conductive hydrogel with medication.

9. According to claim 1,
the patch device, wherein at least a part of a plurality of the adhesion portions comprises a medication available for iontophoresis.

10. According to claim 1,
the patch device, further comprising a medication pouch supplying medication, connected to at least a part of a plurality of the adhesion portion.

11. According to claim 10,
the patch device, wherein the amount of medication supplied by the medication pouch is controlled depending on the amount of the current transmitted by a plurality of the adhesion portions, or on the brainwave received from a plurality of the adhesion portions.

12. According to claim 1,
the patch device, wherein the cover layer is formed with an insulating material.

13. According to claim 1,
the patch device, wherein the stimulation control circuit is formed only with a current limiting diode.

14. According to claim 7,
the patch device, further comprising a flexible power supply portion formed on the first side and covered by the cover layer.

15. According to claim 7,
the patch device, wherein at least a part of a plurality of the adhesion portions comprises a flexible power supply portion.

16. According to claim 7,
the patch device, further comprising a plurality of electrode portions, the surface of which covered with a coating layer made of conductive and
non-invasive solid, wherein a plurality of the adhesion portions are formed on a plurality of the electrode portions.

17. According to claim 2,
the patch device, wherein at least a part of a plurality of the adhesion portions comprises a conductive hydrogel.

18. According to claim 2,
the patch device, wherein at least a part of a plurality of the adhesion portions comprises a medication available for iontophoresis.

19. According to claim 2,
the patch device, further comprising a medication pouch supplying medication, connected to at least a part of a plurality of the adhesion portion.

20. According to claim 2,
the patch device, wherein the cover layer is formed with an insulating material.
